(19) United States
(12) Patent Application Publication
(10) Pub. No.: US 2006/0063260 A1
(43) Pub. Date: Mar. 23, 2006

(54) Method for producing a support carrying immobilized viruses, and the use of such a support

(75) Inventors: Hansjuergen Volkmer, Tuebingen (DE); Frank Weise, Kusterdingen (DE); Thomas Joos, Tuebingen (DE)

Correspondence Address: MORRISON & FOERSTER LLP, 12531 HIGH BLUFF DRIVE, SUITE 100, SAN DIEGO, CA, 92130-2040, US

(73) Assignee: NMI Naturwissenschaftliches und Medizinisches Institut an der Universitaet Tuebingen

(21) Appl. No.: 11/219,122

(22) Filed: Sep. 2, 2005

Related U.S. Application Data
(63) Continuation of application No. PCT/EP04/02054, filed on Mar. 2, 2004.

(30) Foreign Application Priority Data
Mar. 4, 2003 (DE) 103 10 252.3
Jul. 9, 2003 (DE) 103 32 117.9

(51) Int. Cl. C12N 15/86; C12N 15/74

(52) U.S. Cl. 435/456; 435/472

(57) ABSTRACT

A method for producing a support possessing a functionalized surface which is suitable for immobilizing viruses and carrying viruses which are immobilized on the functionalized surface and which are preferably infectious, as well as the use of such supports, are described. In this connection, a support material is initially provided and at least one surface of the support material is then coated, preferably with a nitrocellulose material, in order to form the functionalized surface. In a following step, a solution which contains viruses, which are preferably infectious, is applied to the functionalized surface, after which the viruses are subsequently enabled to adhere to the functionalized surface over a period of from preferably 10 minutes to 36 hours. After that, the surface is, where appropriate, covered with a fluid.

### RELATED APPLICATION

This is a continuation of copending International Patent Application PCT/EP2004/002054 filed on Mar. 2, 2004 and designating the US, which has been published in German as WO 2004/078906 A2 and which claims priority of German Patent Applications Nos. 103 10 252.3, filed on Mar. 4, 2003 and 103 32 117.9, filed on Jul. 9, 2003.

### BACKGROUND OF THE INVENTION

1. Field of the Invention

The invention relates to a method for producing a support possessing a functionalized surface and, in addition, to supports which are produced by this method.

2. Related Prior Art

Supports possessing a functionalized surface are used, in particular, in the fields of medicine and bioanalysis as well as, in particular, in those instances where there is a need for rapid, reproducible and sensitive analytical methods. Methods for producing supports possessing functionalized surfaces are adequately disclosed in the prior art.

Supports possessing functionalized surfaces nowadays play a prominent role for analytical methods in biology and for medical diagnosis, in particular. The supports are miniaturized, microstructured functional elements which possess a large number of biological and/or technical components, e.g. which possess biomolecules which are immobilized on a surface and which can serve as specific interaction partners, as well as possessing a matrix or a support material. In this connection, the “functionalized” surface of the support normally exhibits molecules which possess functional groups which bind to “trapping molecules” which interact with other (bio)molecules, i.e. the “ligands”.

In this connection, a large number of biomolecules are arranged on the support surfaces such that a large number of different (e.g. genetic) items of information can be investigated in parallel and from one sample.

Biomolecules which are arranged on the supports as these binding agents have become an important tool in biotechnology. Examples of trapping molecules which are arranged on supports as “spots” are nucleic acids, such as DNA or RNA, and, in addition, proteins or pep-tides, such as antigens, antibodies and receptors, polysaccharides, lectins or even whole cells, with these substances being arranged in a particular arrangement (“array”) on a solid support surface or in a virtual array on addressable beads. These arrays can, for example, be employed in gene discovery, genome research, diagnosis and bioanalysis and in connection with screening for novel bioactive compounds. An important use of the arrays is in the analysis of differential gene expression, in which analysis, the expression of genes in different cells typically the cell to be investigated, is compared with a control. The differences in the expression of different genes are identified in this context. It is furthermore important to measure many different ligands derived from small quantities of sample.

The said arrays, and/or their use, are accordingly modern methods which can be used to carry out an extensively automated and miniaturized analysis of individual processes in parallel.

Another example of the use of the abovementioned supports carrying immobilized bio-molecules is that of using these supports for detecting, isolating or characterizing cells in a solution. Thus, for example, an investigation can be carried out to determine which cells in a sample bind to give certain immobilized biomolecules and which do not, with it being possible, for example, to employ nucleic acids or proteins as biomolecules.

Another possibility for using supports possessing immobilized biomolecules in biotechnology is, for example, the method as described by Sabatini in US Patent Application U.S. 2002/0006664. This publication discloses a method and a support in connection with which DNA fragments and DNA which is cloned in plasmid vectors are initially arranged and immobilized, as spots, on a support surface. In a next step, cells are sown on this support surface and the DNA which is immobilized on the support surface is then inserted into the cells, a process which is described by the inventors of this method as being “a reverse transfection process”.

However, a major disadvantage of this known method of carrying out reverse transfection is that the cells colonize the support surface outside the spots as well as binding to the immobilized spots. It is therefore always necessary, in the prior art, to identify the cells, in an additional step, which have come into contact with the genetic material.

A further disadvantage is to be seen in the fact that it is only possible to use cells which are easy to transfect with plasmids.

Furthermore, there is generally a serious risk, in this method, that it will not be possible to selectively isolate particular cells from a cell mixture since different cells will be able to bind between the spots.

### SUMMARY OF THE INVENTION

Against this background, an object of the present invention is to provide a support carrying novel biomolecules as well as a method for producing it, which method makes it possible to overcome the abovementioned disadvantages.

According to the invention, this object is achieved by means of a method for producing a support possessing a functionalized surface and carrying viruses which are immobilized on the functionalized surface and which are preferably infectious, which method exhibits the following steps:
(a) providing a support material;
(b) coating at least one surface of the support material for the purpose of producing a functionalized surface which is suitable for immobilizing viruses;
(c) applying a solution, which preferably contains infectious viruses, to the functionalized surface;
(d) leaving the viruses to adhere to the functionalized surface over a period of from preferably 10 minutes to 36 hours;
(e) where appropriate, covering the surface with a fluid.

In addition, the object is achieved by means of a support possessing a functionalized surface which is suitable for immobilizing viruses and carrying viruses which are immobilized on the functionalized surface and which are preferably infectious.

This thereby achieves one object underlying the invention in full.

In their own experiments, the inventors were able to produce a support which possessed a functionalized surface which was suitable for immobilizing viruses and which carried viruses which were immobilized on the functionalized surface, with the support being able to be used to immobilize cells selectively on spots on the support material. In this connection, the inventors were able to demonstrate that the support, and/or the immobilization of the viruses, proved to be stable and that the viruses were still infectious, and able to bind cells, over a relatively long period of time.

It has hitherto been impossible, in the prior art, to immobilize infectious viruses on surfaces of a support. On the contrary, it has generally been considered that the storage or immobilization of viruses, other than in infected cells, over periods lasting many days was unstable even at low temperatures and consequently impossible (see, for example, Davis et al., “Microbiology”, 3rd edition, 1985, p. 1049). It is now possible to use the method according to the invention to produce supports on which infectious viruses are immobilized. In this connection, the inventors were furthermore able to demonstrate that the supports which are produced in accordance with the invention can be stored for a relatively long period at room temperatures and remain serviceable, i.e. the viruses retain their binding ability and infectivity.

Supports carrying viruses which are immobilized in this way and which are still able to bind to cells after the immobilization have not previously been disclosed in the prior art. Even arrays of non-infectious viruses offer many novel possible applications in which the binding between viruses and cells is investigated.

A further advantage of the novel supports is that the viruses make possible gene transfer into, or the transduction of, cells which are difficult to transfect or which cannot be transfected at all. These are cells, for example, which are highly relevant, such as primary cells, for example. It is consequently possible to use the supports which have been produced in accordance with the invention to increase the spectrum of cells into which genes can be transferred markedly as compared with the plasmid transfection in accordance with Sabatini.

The novel supports make it possible for cells to be able to bind, by way of surface receptors, to immobilized viruses, which latter have in turn the ability to infect these cells. When the virus suspension is applied in rows and columns in array form, cells can be infected in miniaturized and parallelized formats. Recombinant immobilized viruses correspondingly permit miniaturized and parallelized gene transfer, as well as gene expression, to take place in cells which can only be manipulated with difficulty by means of other recombinant methods.

In this connection, the viruses are arranged in individual spots, with different spots being able to contain different virus types or species. The consideration which is of importance for the transduction of cells in this connection is the PFU, that is the plaque forming units, which specifies how many infectious virus particles are present in a spot.

Using the novel supports, it is also possible, for the first time, to provide arrays of infectious, and also non-infectious, viruses which are able to specifically bind different cells. In this connection, it is also possible to immobilize different virus types and species whose ability to bind to cells from a sample is investigated, with it being possible, on account of the miniaturization and parallelization, to work with small quantities of sample.

Within the context of the present application, “support” denotes both a planar support possessing a physically coherent surface and a virtual array composed of beads or microbeads which can furthermore be labelled and consequently be addressable.

“Support material” denotes, in the present case, a support possessing at least one surface on which biomolecules such as nucleic acids, proteins, cells or viruses can be immobilized in a general manner. The skilled person will recognise that a large number of support materials which are employed in the prior art, for example in the field of bioanalysis, are suitable for the method according to the invention.

“Functionalized surface” denotes, in the present case, any surface which exhibits molecules possessing functional groups by way of which the binding of biomolecules, for example nucleic acids, proteins, whole cells, etc. is mediated.

“A functionalized surface which is suitable for immobilizing viruses” denotes, in the pre-sent case, a surface possessing a coating which, on the one hand, binds, that is immobilizes, the viruses but which, on the other hand, makes it possible for the virus particles in or at the surface to be still readily accessible for cells. In other words, the viruses are still so flexible that they are able to sterically reach the cell surface receptors and do not mutually impede each other in doing so. According to the inventors' findings, the functionalized surface makes this possible in particular when it forms a type of meshwork in which the virus particles are bound firmly but flexibly. According to the inventors' surprising findings, this can be achieved, for example, using a coating composed of nitrocellulose or materials possessing similar properties, where the virus particles are, for example, bound, by way of an electrostatic interaction, to individual fibres which exhibit a certain flexibility in the coating. However, it is also possible to employ other mechanisms which permit the immobilized viruses, which can be present as aggregates, to bind to the cells. In the case of a desired gene transfer, this binding must, where appropriate, permit the viruses to be internalized.

For this, the virus particles should be bound non-covalently, as is achieved, for example, with a coating composed of nitrocellulose, which is, where appropriate, additionally coated with collagen, or with coatings which give rise to a lasting positive charge on the surface, as is the case, for example, with the commercially obtainable “Superfrost (TM) Plus” supports which are marketed by EMS, Electron Microscopy Sciences in . . . . It is furthermore possible to use aminosilane-coated surfaces as is the case, for example, with the commercially obtainable “GAPS II Coated Slides” which are marketed by Corning Inc., Acton, Mass., USA.

“Infectious” denotes, in the present case, that, before and after the immobilization, the vi-ruses are able to infect cells, that is, on contact with cells, to insert into the cells the genetic material, i.e. DNA or RNA, which is contained in the viruses, if this is the aim of using a support which is produced by the method according to the invention. In this connection, care has also to be taken to ensure that the PFU is sufficiently high in a spot so. that a sufficient number of binding points for the adhesion of the cells which are to be immobilized on immobilized viruses are present.

On the other hand, “noninfectious” means that the viruses are able to bind cells without, however, inserting their genetic material into the bound cells.

The support according to the invention can be used for a variety of purposes, in particular for investigating the interaction, with the immobilized viruses, of cells which are present in a solution. When a support which has been produced by the method in accordance with the invention is used, it is possible after the support has been incubated with a cell-containing solution, for these cells to be focused on spots on the support, that is only to bind to sites at which viruses are immobilized.

The inventors were furthermore able to demonstrate, in their own experiments, that it was possible, by way of the stably immobilized viruses, to introduce foreign genetic material into cells which bind to the viruses. This demonstrated that, as a result of the method according to the invention, the viruses remain infectious even in the immobilized state.

After the viruses have been immobilized on the support surface, the support can, to pre-vent drying out, be covered with a suitable fluid, for example with phosphate-buffered salt solution (PBS), polyethylene glycol (PEG) or solutions of protein or polysaccharide.

According to another object the support material exhibits a material which is suitable for use as a surface for cell cultures and is preferably selected from the group comprising cell culture plastics, glass, silicone and polystyrene.

These support materials have proved their worth in the prior art and are used in many different ways in this field. Consequently, within the meaning of the present invention, “support material” can be any material which is suitable for implementing this method and which is used in the prior art for producing similar supports, for example labelled microbeads and planar cell culture supports as well.

According to another object the support material is precoated, in the method, with a polycation, for example with poly-L-lysine or aminosilane, with aminosilane also itself already being a surface which is suitable for immobilizing viruses.

Surfaces which are coated in this way are adequately disclosed in the prior art and are also frequently used in medical fields. Thus, it is possible, for example, in the method according to the invention, to use, as the support material, a glass microscope slide which has been pre-coated with poly-L-lysine.

Glass microscope slides are, for example, marketed by the Sigma company, Taufkirchen, Germany, under the name PolyPrep.

According to another object the solution which contains the viruses also contains glycerol.

The use of glycerol has the advantage that the viruses are kept active in the moist state. Apart from glycerol, it is also possible to conceive of using glycerol-like compounds, such as trehalose, sucrose and, in a general manner, substances which can stabilize protein solutions, in the method.

Preference is furthermore given to the viruses which are present in the solution being adenoviruses or adeno-associated viruses (AAVs). In this connection, preference is given, in particular, to serotype 5 adenoviruses.

The inventors have found that it is possible, for example by using serotype 5 adenoviruses, to focus cells on the viruses which are immobilized on the support material. It has previously been considered, in the prior art, that it was not possible to store or immobilize infectious adenoviruses outside cells or living hosts.

In a further development of the method according to the invention, preference is given to the viruses being altered recombinantly.

This means that virus-foreign sequences can be incorporated into the genetic information of the viruses, with these sequences being expressed in the immobilized cells. In this connection, the recombinant change in the virus genome can be carried out using methods which are customary in the prior art. The viruses are then so called vectors, with the viruses in this connection being selected from the group: adenoviruses and AAVs.

Preference is given, in particular, to the viruses exhibiting a sequence which encodes the green-fluorescent protein. This protein was cloned for the first time by Prasher et al., “Primary structure of the Aequorea victoria green-fluorescent protein”, Gene 111: 229-233 (1992), and has proved to be an outstanding reporter gene in a very wide variety of areas of biology and medicine, with the gene indicating whether genetic material has been transferred from the viruses into the cells.

Other examples of suitable reporter genes are β-galactosidase, luciferase, alkaline phosphatase and peroxidase.

Preference is furthermore given to a supernatant from virus-infected cells, and, in particular a supernatant which is purified and/or concentrated before being applied to the functionalized surface, being used as the virus-containing solution.

In their own experiments, the inventors were able to demonstrate that the immobilization proved to be particularly stable when the virus solution which was obtained from the supernatant from infected cells was purified and/or concentrated before being applied. The virus material, or the cell culture supernatant, can, for example, be purified by means of a density gradient centrifugation, in particular a caesium chloride (CsCl) density gradient centrifugation. Despite the above-described crude treatment, the purified and subsequently immobilized viruses were also still infectious even over a relatively long period of time. This was not to be expected in accordance with the prior art.

In one embodiment of the method according to the invention, preference is given to bovine serum albumin being applied to the functionalized surface prior to step (c).

The inventors found that this step advantageously prolonged the stability and infectivity of the viruses still further.

In a further development of the method according to the invention, preference is given to the period in which the viruses adhere to the functionalized surface being from 15 to 20 hours.

While this period, which was selected by the inventors in a number of experiments, proved to be suitable for the viruses to become adequately adhered, shorter periods are also possible if the method is to be speeded up.

Preference is furthermore given, in the method according to the invention, to the temperature at which the viruses adhere to the functionalized surface being from −20° C. to +37° C. and, in particular, from 0° C. to +10° C.

In this connection, a low temperature range was selected, in the inventors' own experiments, in order to ensure the stabilization, and thus the infectivity, of the viruses. In this connection, the temperature is preferably above the freezing point of the suspension.

Another object of the invention refers to supports possessing a functionalized surface which is suitable for immobilizing viruses and carrying viruses which are immobilized on the functionalized surface and which are preferably infectious, and, in particular, to those supports which are produced by the method according to the invention.

A further object of the invention relates to a method for investigating medical samples comprising the steps of bringing into contact a medical sample with a support on whose surface viruses are immobilized and analyzing the reaction between substances which are present in the medical sample and the viruses which are immobilized on the support surface.

This method offers the advantage that medical samples can be treated with, for example, virus-specific detection molecules, as a rule antibodies. This reaction can then be analysed for binding to the immobilized viruses within the context of a high-throughput process.

In this connection, “medical sample” is understood as being a sample which contains cells, antibodies or, in a general manner, proteins which are relevant for diagnostic or therapeutic purposes and whose ability to bind to the immobilized viruses is to be tested.

In this connection, an area in which the novel supports can be used is that of screening libraries of recombinantly altered viruses, in particular adenoviruses, for the ability of the viruses to adhere to cells. In this connection, these viruses can be screened for an altered tropism when, that is, the envelope proteins are altered genetically in order to make the viruses specific for a new target tissue. These viruses are employed, in particular, in gene therapy when genetic material is to be inserted into particular target tissues and when the neighbouring tissue is not to be accessible for the transduction.

When the viruses are phages, the novel supports can also be used for phage display. In phage display, different antibody fragments, for example, whose ability to bind to cells or proteins is being tested, are expressed on the shell of bacteriophages.

A further object of the invention relates to a method for characterizing cells and/or investigating cell adhesion comprising the steps of bringing into contact a cell suspension with a support on whose surface viruses are immobilized and analyzing the reaction of the cells which are present in the suspension with the viruses.

In this case, the cell adhesion is tested, for example, either for particular cells against many different viruses or for particular viruses against many different cells.

The characterization of cells is important for analytical problems, in particular. In this connection, an advantage of the method according to the invention is the specificity of the interaction of the viral ligands with the cellular receptors. Thus, the cells to be investigated, or the cells which bind to the immobilized viruses, can be enriched without difficulty from a cell mixture even in the case of small initial quantities.

In procedures taking place downstream of this method, the enriched cells can be employed for a large number of further investigations such as, for example, investigating metabolism or the reaction to external stimuli, environmental influences, etc.

Another object of the invention relates to a method for transferring genetic material into cells.

In this case, gene transfer is also tested or utilized in addition to the abovementioned cell adhesion.

Particular preference is given to this method when the following steps are carried out:
(a) providing a support possessing a functionalized surface which is suitable for immobilizing viruses and carrying infectious viruses which are immobilized on the functionalized surface, and
(b) bringing the support into contact with a suspension which contains cells.

This method offers the advantage that it enables automated, miniaturized, parallel gene transfer to take place. The immobilization of viruses makes it possible to transfect primary cells as well with genetic material. Genes which do not originate from the viral gene repertoire are also, for example, of interest in this connection: thus, for example, gene material can be inserted into the viruses, the viruses can then be immobilized on the support surface and the cells to be transfected can then be added to the support.

The method according to the invention provides the possibility of introducing any genetic information into the target cells since, after the cells have adhered to the immobilized viruses, the latter insert their gene material into the adhering cells. Advantageously, this thereby only transfects the cells which are also adhering to immobilized viruses. A method of this nature is of importance, in particular, for cell types which are not accessible to classical physical gene transfer.

The gene transfer can be detected, for example by the reaction of the cell to the gene transfer, using a variety of tests, for example visually by way of changes in the morphology, enzymically by way of changes in the metabolism, immunologically by way of a change in the cell products characterizing the cell status, by way of the expression of reporter genes, etc.

If viruses which have been altered recombinantly are used instead of the naturally occur-ring viruses, the possibility then exists of introducing any genetic information into the cell.

While the transfection of cells with DNA has already been described by Sabatini et al. (see US Patent Application U.S. 2002/0006664 A1), the cells are in this case transfected by employing DNA which is present in an expression vector, with use being made of a mixture containing a carrier protein (preferably gelatine), which mixture is immobilized on the support material. The document does not describe, or propose, immobilizing viruses which contain DNA which is to be transduced.

Preference is given, in embodiments of the methods according to the invention, to the cells being eukaryotic cells, in particular primary cells or immortalized cells.

Especially in the case of primary cells, the method according to the invention for gene transfer is an outstanding method for transducing these cells since, as is known, it is difficult to transfer genes into these cells using conventional methods.

On the other hand, it is also possible to immobilize bacteriophages and transduce bacteria, for example. A method which may be mentioned in this connection is that of monitoring expression when screening for gene transfer having taken place. However, it is also possible to investigate libraries of phages for their ability to bind to cell surface receptors, as can be employed for phage display, for example.

In a general manner, preference is given to making use of a support, for the methods according to the invention, which has been produced by the abovementioned method according to the invention.

The invention furthermore relates, in a general manner, to a method for inserting DNA or RNA into a prokaryotic or eukaryotic cell, involving the steps of:
(a) applying a virus-containing solution to a support possessing a functionalized surface which is suitable for immobilizing viruses,
(b) allowing the solution to adhere to the surface, and
(c) applying a suspension, which contains cells, to the surface in order to transduce the cells with the DNA or RNA which is contained in the viruses.

The method according to the invention offers the outstanding possibility of rapidly and efficiently inserting genetic material into cells. This method is more advantageous as compared with the known method, in which the DNA to be transfected is immobilized directly on the support, since the cells are in the present case specifically focused on the spots, i.e. only bind to sites on the support on which the viruses are immobilized.

In this connection, particular preference is given to making use of a support which has been produced by the method according to the invention.

By means of their own experiments, the inventors were able to demonstrate that supports which have been produced in accordance with the invention and which are carrying immobilized viruses can be used to insert genetic material into cells which are added, for example in a suspension, to the support. A method of this nature, and a support to be used for this method, were not previously known in the prior art since the immobilization of viruses, and stable storage, were not previously possible.

Another object of the invention relates to a method for storing viruses which are preferably infectious on a support possessing a functionalized surface which is suitable for immobilizing viruses and, in particular, on a support which has been produced by the method according to the invention.

In their own experiments, the inventors were able to demonstrate that it is possible to stably immobilize viruses on a support for a relatively long period without the viruses losing their ability to bind to cells or losing their infectivity. As mentioned above, storing infectious viruses outside cells or corresponding living hosts was not previously known in the prior art.

It will be understood that the features which are mentioned above, and those which are still to be explained below, can be used not only in the combination which is in each case specified but also on their own without departing from the scope of the present invention.

### BRIEF DESCRIPTION OF THE DRAWINGS

Embodiments of the invention are presented and explained in the following examples and the attached figures.

FIG. 1a shows a diagram of a perspective view of a support according to the invention.

FIG. 1b shows a diagram of an overhead view of the support according to the invention from FIG. 1a.

FIG. 2a shows fluorescence-microscopic photographs of infected and noninfected HEK293 cells after 7 days.

FIG. 2b shows fluorescence-microscopic photographs of infected and noninfected HEK293 cells after 14 days.

FIG. 3 shows a collage of different sectors of the fluorescence of a spot in a chamber.

FIG. 4 shows fluorescence-microscopic photographs of infected primary cells.

FIGS. 5-9 show photographs as in FIGS. 2a and 2b but for other surfaces.

### DESCRIPTION OF PREFERRED EMBODIMENTS

### EXAMPLE 1

### Functionalizing the Surface of the Support Material

Commercially obtainable glass microscope slides (PolyPrep, Sigma, Taufkirchen, Ger-many) which were pre-coated with poly-L-lysine were coated with nitrocellulose. For this, the slides were inserted for 2 minutes into a dipping bath which was filled with nitrocellulose (2.5 mg/ml) dissolved in methanol. The slides were then dried under sterile conditions. In this way, the functionalized surface was prepared for the subsequent immobilization of the viruses.

Immobilizing the Viruses

a) Describing the Virus Model Employed

Commercially obtainable serotype 5 adenoviruses (AdEasy-1, Stratagene, La Jolla, USA), which, as a result of recombinant alteration, are dependent for their replication on special auxiliary cells (HEK293), were used as a model system. While these viruses display the property of being able to infect a broad spectrum of mammalian cells, they are not able to replicate in these cells. A further recombinant alteration was used to introduce, into these viruses, a foreign gene which yields a product, i.e. the green-fluorescent protein (termed GFP below), which can be detected by fluorescence. Both the virus and GFP are used as a standard feature in molecular biological laboratories. In that which follows, the virus employed is termed AdEasy-CMV-GFP.

b) Preparing the viruses

The following virus preparations were employed by the inventors: on the one hand, virus-containing supernatant from infected HEK293 cells was used while, on the other hand, viruses which were purified from this cell supernatant by means of a density gradient centrifugation (caesium chloride gradient) were used. HEK (human embryonic kidney) 293 cells can be obtained, for example, from the Deutsche Sammlung für Mikroorganismen und Zellkulturen [German Collection of Microorganisms and Cell Cultures] (DSMZ) under the DSMZ number ACC305. The two virus preparations were in each case diluted 1:1.3 with loading buffer. The loading buffer is an 80% solution of glycerol in phosphate-buffered salt solution (PBS).

C) Immobilizing the Viruses in an Array Arrangement

In the model experiment, 8 loadable chambers were installed on the slides (see above) by gently pressing disinfected plastic gratings (FlexiPERM, Vivascience, Göttingen) onto the surface. In order to prevent contacts between the chambers, the bearing surface of the gratings was previously impregnated with petroleum jelly (Vaseline, Lever Faberge, Hamburg).

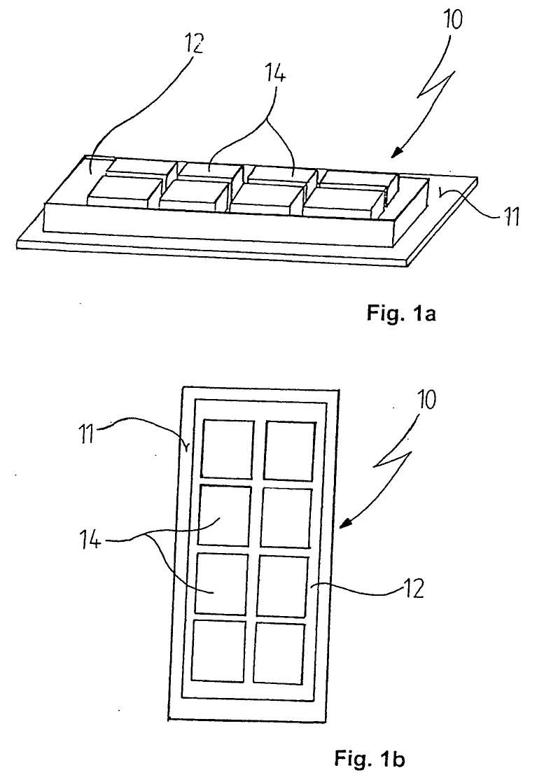

FIG. 1 depicts a diagram of an embodiment of the slide according to the invention, with FIG. 1a depicting a perspective view and FIG. 1b depicting an overhead view onto the slide. In the figures, the same elements are labelled with the same reference numbers. In FIGS. 1a and 1b, a slide is depicted in a general manner by the reference number 10, with the slide having a surface 11 and having a grating attachment 12 which is applied to the surface 11. Before the grating attachment 12 was applied, the surface 11 of the slide 10 was coated with nitrocellulose. On its underside, the grating attachment 12 is provided with a layer of vaseline. Applying the grating attachment 12 to the nitrocellulose-coated surface 11 results in the formation of chambers 14, contact between which is prevented by the vaseline layer under the grating attachment 12.

The suspended viruses were loaded (loading volume: 1 μl) onto the centre of the cham-bers 14. Because of the gravitational force acting on them, the viruses sediment from the suspension onto the functionalized surface, where they adhere. In order to allow sufficient time for the viruses to sediment and adhere, the slides were kept at 4° C. for 18 hours. After that, the chambers were filled with 500 μl of PBS in order to prevent any drying out. The slides were then stored at 37° C.

Before sowing the cells, the chambers were washed with 500 μl of PBS in order to re-move any viruses which might possibly have become resuspended.

### EXAMPLE 2

### Stability of the Immobilized Viruses

For practical applicability, it was important to investigate the degree to which the immobilized viruses were stable under normal environmental conditions since it is as a rule to be assumed that the requirement is for the immobilization of the viruses and the colonization of the cells to be separated spatially and chronologically. For this reason, the ability of the viruses which are immobilized using the methods according to the invention to be transported and stored is an outstanding feature.

For the experiment, viruses (AdEasy-CMV-GFP) were immobilized and, following a 7-day or a 14-day storage at 37° C., 105 HEK293 cells were in each case sown once per chamber. The infection which had taken place was detected, in each case one day after the cells had been sown, by means of taking fluorescence-microscopic photographs: infected cells, which had taken up the GFP gene, were excited by laser light to fluoresce.

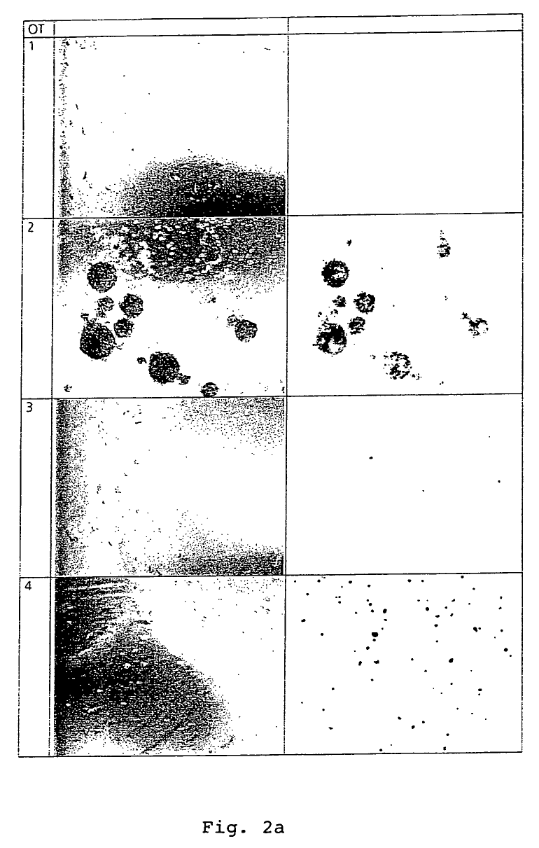

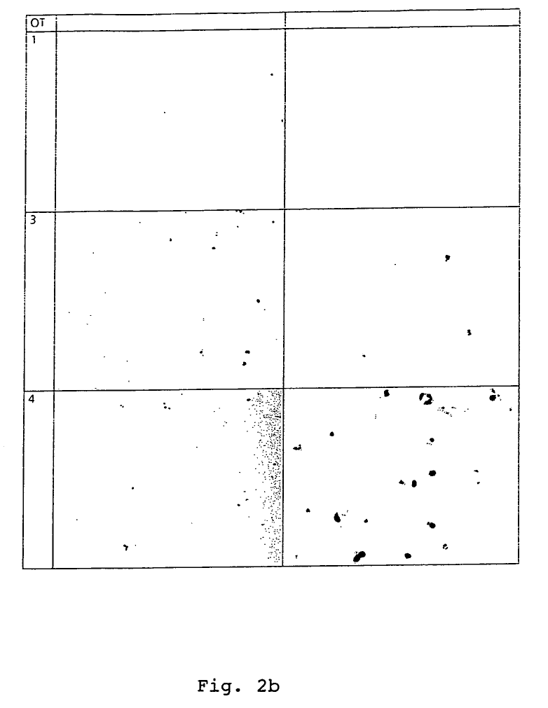

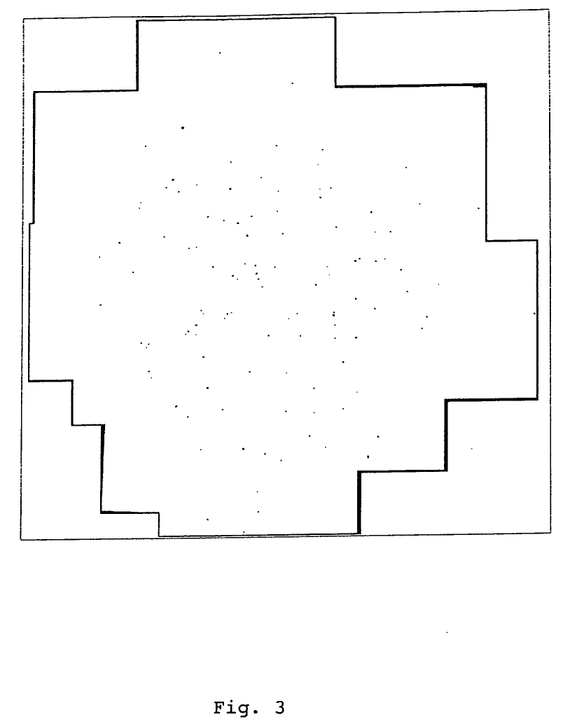

FIGS. 2a and 2b depict the fluorescence-microscopic photographs from this experiment, with in each case four slides being depicted in transmitted light photographs in the left-hand column and being depicted in fluorescence photographs in the right-hand column. The fluorescence photographs are inverted for better portrayal.

TABLE 1
Experiment 1
Stability of the immobilized viruses

| | | Cell | | Cell | |
|---|---|---|---|---|---|
| | Virus | loading | Fluorescence | loading | Fluorescence |
| Slide | suspension | Day 7 | Day 8 | Day 14 | Day 15 |
| 1 | −−− | HEK293 | − | HEK293 | − |
| 2 | supernatant | HEK293 | + | HEK293 | − |
| 3 | CsCl | HEK293 | +++ | HEK293 | + |
| 4 | CsCl/BSA | HEK293 | +++ | HEK293 | + |

Four different assays were carried out for the experiment: only loading buffer, without any virus suspension, was loaded onto one slide No. 1 (negative control). Different preparations of viruses were immobilized on slides Nos. 2 to 4: the supernatant from infected HEK293 cells was added to slide No. 2, while viruses which had been purified through a caesium chloride gradient were added to slide No. 3 and viruses where bovine serum albumin (BSA, 0.5 μl, 1 mg/ml) had been initially introduced onto the spot were added to slide No. 4.

Table 1 shows the results of this experiment. Adenoviruses from the different preparation steps were immobilized in the chambers and the chambers were loaded with cells at the specified times (on day 7 and on day 14 after the immobilization). In the case of caesium chloride/BSA, 0.5 μl of bovine serum albumin (1 mg/ml) was introduced initially, after which 1 μl of suspension was loaded on. In Table 1, the relative fluorescence intensities are classified by the following symbols:
− no fluorescence
+weak fluorescence
+++ strong fluorescence.

In the case of viruses which had been isolated from the supernatant from infected HEK293 cells, but which had not been further purified, it was still possible to observe fluorescence after 7 days of storage but not, however, after the viruses had been stored for 14 days (see slide 2, cell loading after day 7 and after day 14; in addition, slide 2 in FIG. 2a and slide 2 in FIG. 2b). The durability or stability of the viruses was markedly improved by purifying them by means of density gradient centrifugation (see slide No. 3 in FIGS. 2a and 2b, and also in Table 1). It was possible to achieve an additional stabilization by initially introducing bovine serum albumin (see slide No. 4 in FIGS. 2a and 2b as well as in Table 1).

The stability of the immobilization per se, i.e. the constancy of the localization of the immobilized viruses on the spot, is evident from FIG. 3, which depicts, for HEK293 cells, a collage of different sectors of the fluorescence of a spot in a chamber on day 1 after the infection: fluorescing cells (inverted for better portrayal) are principally located, as can be seen in the figure, within the circular spot area.

It can therefore be concluded from this experiment that the viruses do not, as a result of their immobilization, lose the ability to infect cells and consequently transfer foreign genetic information. It was furthermore found that, when suitable immobilization conditions are chosen, the infectivity persists for at least two weeks. In addition, the method ensures the immobilization of the viruses, and the stability of their localization, within the original spot area.

The inventors were therefore able to demonstrate that immobilized adenoviruses were stable for at least 14 days at 37° C. and enabled HEK293 cells to be infected, including transfer and expression of the reporter gene GFP.

### EXAMPLE 3

### Infecting, Primary Cells with Immobilized Viruses

As mentioned above, the advantage of using viruses as a means of gene transfer consists in the spectrum of manipulatable cell types being markedly wider than in the case of the classical physical methods. These cells which can only be manipulated efficiently using viruses include many primary cells, that is cells which either cannot be cultured or lose their characteristic properties during culture. For true-to-life investigations, these cells always have to be isolated freshly from the tissue, with this resulting in a radical restriction in the quantity of cells available.

Microglia cells from the brains of 3-day-old rats were used as examples of primary cells in a following experiment. Adenoviruses (AdEasy-CMV-GFP) were applied to coated slides, and immobilized, as described above. After 8 days, cells (HEK293 as the positive control, microglia as the primary cells to be tested) were sown at the rate of 5×10⁴ per chamber.

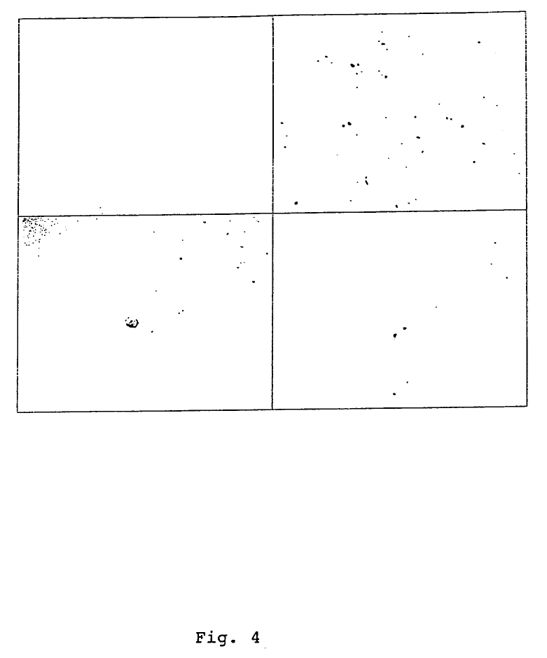

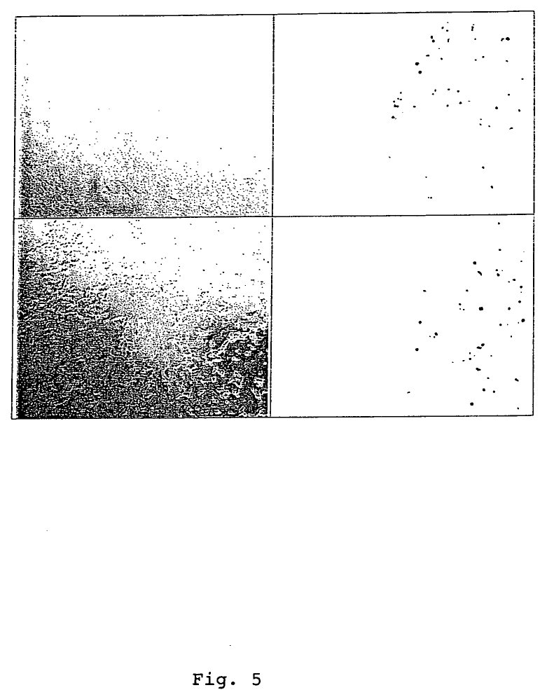

After two days, fluorescence-microscopic photographs were taken (see FIG. 4). The upper row shows transmitted light (left-hand column) and fluorescence (right-hand column) photographs for HEK293 cells while microglia cells are shown in the lower row. The exposure time in the case of the fluorescence photographs was 5 seconds. As already demonstrated in the abovementioned experiment, strong fluorescence was also seen in the present case when the HEK293 cells were excited with laser light. It was furthermore also possible to see isolated fluorescing cells in the case of the microglia cells.

This experiment demonstrated that it was also possible to infect microglia cells, just like the control cells, with the immobilized viruses. This thereby demonstrated that the method was also applicable in principle in the case of primary cells, which can only be accessed with difficulty using the classical physical gene transfer methods.

### EXAMPLE 4

### Estimating the Efficiency with which Immobilized Viruses Infect Primary Cells

In the case of many test systems, it is important, particularly when the quantity of cells to be tested is small, that the genetic information which is mediated by the viruses should reach as many cells as possible because the effects to be measured could otherwise fall below the detection limit. It is therefore of fundamental importance that the infection should succeed in the overwhelming majority of the cells and not just in a minority. The efficiency of the infection depends, inter alia, on a sufficiently large number of immobilized viruses retaining their infectivity.

In order to investigate the question of infection efficiency, the experiment of infecting microglia cells was repeated as described above. All the cells which were visible as a result of taking up light were first of all counted, at 200-fold magnification, on two randomly selected image sectors, after which the cells which were fluorescing as a result of having been excited by laser light were counted on the same sectors.

The count showed that, in one case, 41 cells out of 66 were fluorescing and that, in the other case, 32 cells out of a total of 54 were fluorescing. As a result, this showed that, in the case of microglia cells, the efficiency with which the reporter gene GFP was transferred and expressed was of the order of size of about 60%.

This showed that a majority of the colonizing primary cells were accessible to the viral gene transfer. This thereby demonstrated that it was possible to use the method under every-day laboratory conditions.

In summary, it was consequently demonstrated that it was possible to immobilize viruses on a small immobilization area (spot) in a grid arrangement on functionalized surfaces and that the infectivity of the viruses was retained for 14 days at 37° C.

The infection by the viruses enabled the foreign gene GFP, which was integrated into the viral genome, to be transferred into cells and expressed in these cells. Successful infection was achieved not only in the case of immortalized cells, as represented by the HEK293 cells, but also in the case of primary cells, with this being at an efficiency of 60%.

### EXAMPLE 5

### Examining Other Surfaces

Other functionalized surfaces were tested, under the conditions described in Examples 1 and 2, for their suitability for being able to immobilize viruses in such a way that the latter remain infectious.

In FIGS. 5-9, the left-hand column in each case once again shows transmitted light photographs while the right-hand column shows fluorescence photographs which have been inverted for better portrayal.

FIG. 5 shows photographs of slides which, while being glass slides which were precoated with poly-L-lysine and then coated with nitrocellulose, as described in Example 1, were also additionally coated with collagen.

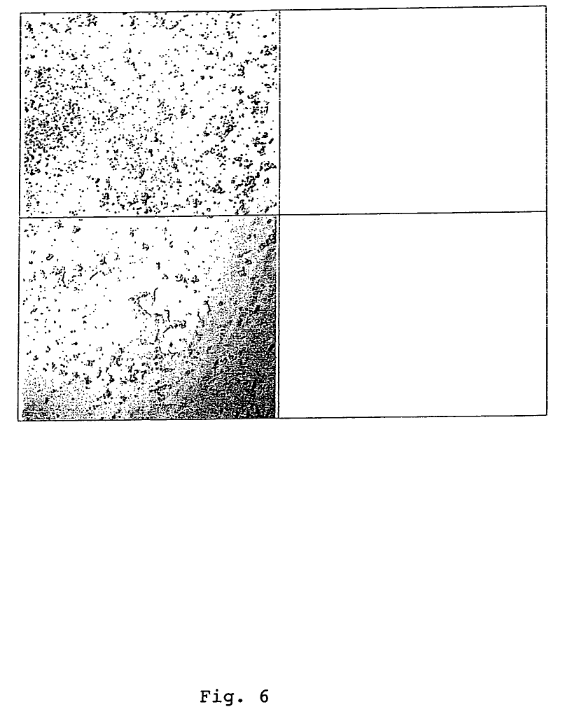

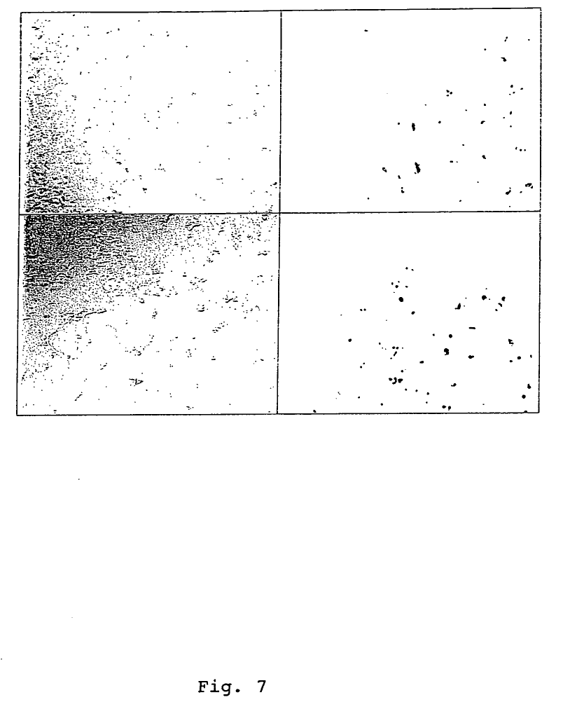

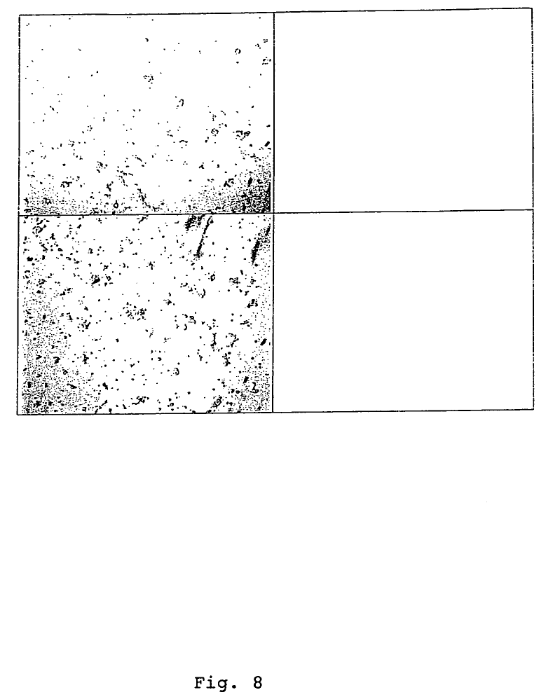

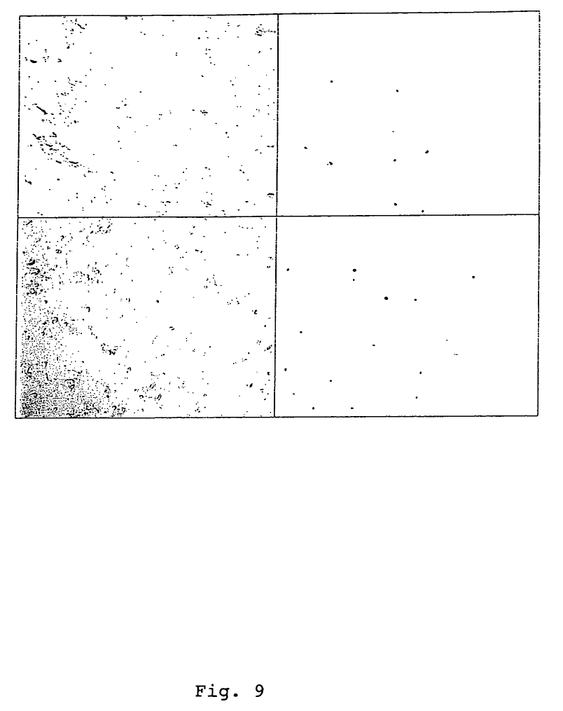

The photographs in FIG. 6 were obtained using silanized aldehyde slides, while the photographs in FIG. 7 were obtained using Superfrost (TM) Plus Adhesion Slides from Electron Microscopy Sciences, the photographs in FIG. 8 were obtained using Spot-On (TM) protein slides from Scandinavian Microbiodevices, and the photographs in FIG. 9 were obtained using GAPS II Coated Slides from Corning.

While it was in each case possible to discern fluorescing HEK293 cells in the right-hand column in FIGS. 5, 7 and 9, this is not the case with the photographs in FIGS. 6 and 8: neither the silanized aldehyde slides in FIG. 6 nor the slides in FIG. 8, in which covalent immobilization is effected by way of epsilon amino groups of the proteins, display any fluorescence.

These results show that the viruses do not have to be coupled covalently in order to be able to remain infectious on the slide.

1. Method for producing a support possessing a functionalized surface and carrying viruses which are immobilized on the functionalized surface and which are preferably infectious, involving the following steps:
(a) providing a support material;
(b) coating at least one surface of the support material for the purpose of producing a functionalized surface which is suitable for immobilizing viruses;
(c) applying a solution, which preferably contains infectious viruses, to the functionalized surface;
(d) leaving the viruses to adhere to the functionalized surface over a period of from preferably 10 minutes to 36 hours;
(e) where appropriate, covering the surface with a fluid.

2. Method according to claim 1, wherein the functionalized surface is coated with a material which binds the viruses noncovalently.

3. Method according to claim 2, wherein the material is selected from nitrocellulose, aminosilane and a material which gives rise to a lasting positive charge on the surface.

4. Method according to claim 1, wherein the support material exhibits a material which is suitable for use as a surface for cell cultures and is preferably selected from the group comprising cell culture plastics, glass, silicone and polystyrene.

5. Method according to claim 1, wherein the support material has been precoated with a polycation, for example with poly-L-lysine or aminosilane.

6. Method according to claim 1, wherein the virus-containing solution, which is applied to the functionalized surface, contains a substance, such as glycerol, trehalose and sucrose, which can stabilize protein solutions.

7. Method according to claim 1, wherein the viruses are adenoviruses or adeno-associated viruses (AAVs).

8. Method according to claim 7, wherein the adenoviruses are of serotype 5.

9. Method according to claim 1, wherein the viruses have been altered recombinantly.

10. Method according to claim 9, wherein the viruses exhibit a sequence which encodes the green-fluorescent protein.

11. Method according to claim 1, wherein a supernatant from virus-infected cells is employed as the virus-containing solution.

12. Method according to claim 11, wherein the supernatant is purified and/or concentrated before being applied to the functionalized surface.

13. Method according to claim 1, wherein bovine serum albumin is applied to the functionalized surface prior to step (c).

14. Method according to claim 1, wherein the period in which the viruses are left to adhere to the functionalized surface is from 15 to 20 hours.

15. Method according to claim 1, wherein the temperature at which the viruses are left to adhere to the functionalized surface is from −20° C. to +37° C.

16. Method according to claim 15, wherein the temperature is from 0° C. to 10° C.

17. Support possessing a functionalized surface which is suitable for immobilizing viruses and carrying viruses which are immobilized on the functionalized surface and which are preferably infectious.

18. Support according to claim 17, that it is produced in accordance with claim 1.

19. Method for investigating medical samples comprising the steps of bringing into contact a medical sample with a support possessing a functionalized surface which is suitable for immobilizing viruses and carrying viruses which are immobilized on the functionalized surface and which are preferably infectious and analyzing binding to the immobilized viruses.

20. Method for characterizing cells and/or investigating cell adhesion comprising the steps of bringing into contact a cell suspension with a support possessing a functionalized surface which is suitable for immobilizing viruses and carrying viruses which are immobilized on the functionalized surface and which are preferably infectious, and analyzing the reaction of the cells which are present in the suspension with the viruses, which cells are selected from prokaryotic cells, eukaryatic cells, primary eukaryotic cells, immortalized cells.

21. Method for transferring genetic material into cells, which cells are selected from prokaryotic cells, eukaryatic cells, primary eukaryotic cells, immortalized cells comprising the following steps:
(a) providing a support possessing a functionalized surface which is suitable for immobilizing viruses and carrying infectious viruses which are immobilized on the functionalized surface, and
(b) bringing the support into contact with a suspension which contains cells.

22. Method according to claim 21, wherein the viruses are bacteriophages.

23. Method according to claim 21, wherein use is made of a support which has been produced according to claim 1.

24. Method for inserting DNA or RNA into a prokaryotic or eukaryotic cell, involving the steps of:
(a) applying a virus-containing solution to a support possessing a functionalized surface which is suitable for immobilizing viruses,
(b) allowing the solution to adhere to the surface, and
(c) applying a suspension, which contains cells, to the surface in order to transduce the cells with the DNA or RNA which is contained in the viruses.

25. Method according to claim 24, wherein use is made of a support which has been produced in accordance with claim 1.

26. Method for storing viruses which are preferably infectious, wherein they are immobilized on a support possessing a functionalized surface.

27. Method according to claim 26, wherein use is made of a support which has been produced in accordance with claim 1.
